SEQUENCE LISTING (47 sequences)

SEQ ID NO: 1 - protein, 20 aa, Artificial Sequence
  Synthetic sequence
GSPERHHKQLKISFSETALE

SEQ ID NO: 2 - protein, 20 aa, Artificial Sequence
  Synthetic sequence
EPPVVKRQKTKVKFDDGQVF

SEQ ID NO: 3 - protein, 21 aa, Artificial Sequence
  Synthetic sequence
CGLERDKFDNKTVTFEEHIKE

SEQ ID NO: 4 - protein, 21 aa, Artificial Sequence
  Synthetic sequence
EKGAGEKRRGKRVRFAEDEEK

SEQ ID NO: 5 - protein, 21 aa, Artificial Sequence
  Synthetic sequence
SNALRNKKNSRRVSFADTIKV

SEQ ID NO: 6 - protein, 20 aa, Artificial Sequence
  Synthetic sequence
PDPETPLKARKVRFSEKVTV

SEQ ID NO: 7 - protein, 20 aa, Artificial Sequence
  Synthetic sequence
HSGSLKKQSPKVTFECEQKE

SEQ ID NO: 8 - protein, 20 aa, Artificial Sequence
  Synthetic sequence
GGRHTHVKRKKVTFLEEVTE

SEQ ID NO: 9 - protein, 21 aa, Artificial Sequence
  Synthetic sequence
EVGVKSEHSGKKVQFAEALAT

SEQ ID NO: 10 - protein, 20 aa, Artificial Sequence
  Synthetic sequence
QRPKRKRKNSRVTFSEDDEI

SEQ ID NO: 11 - protein, 21 aa, Artificial Sequence
  Synthetic sequence
IPLECPLSSPKGVLFSSKSAE

SEQ ID NO: 12 - protein, 21 aa, Artificial Sequence
  Synthetic sequence
LPSQNRLQPQKHVSFTPGDDM

SEQ ID NO: 13 - protein, 21 aa, Artificial Sequence
  Synthetic sequence
IMLGNKKKKKKNVKFPDEDEI

SEQ ID NO: 14 - protein, 21 aa, Artificial Sequence
  Synthetic sequence
PSNQKCGGRKKTVSFSSMPSE

SEQ ID NO: 15 - protein, 21 aa, Artificial Sequence
  Synthetic sequence
ERKRSTGGRRKKVSFEASVAL

SEQ ID NO: 16 - protein, 20 aa, Artificial Sequence
  Synthetic sequence
KGSEEAKGRFRVNFVDPAAS

SEQ ID NO: 17 - protein, 21 aa, Artificial Sequence
  Synthetic sequence
KTPIKKPGDGRKVTFFEPGSG

SEQ ID NO: 18 - protein, 21 aa, Artificial Sequence
  DGSGGARAKSKSVSFNLGCKK
DGSGGARAKSKSVSFNLGCKK

SEQ ID NO: 19 - protein, 21 aa, Artificial Sequence
  Synthetic sequence
PPPQVVQMYGRKVGFALDLPP

SEQ ID NO: 20 - protein, 20 aa, Artificial Sequence
  Synthetic sequence
TGSERIAHGMRVKFNPLALL

SEQ ID NO: 21 - protein, 20 aa, Artificial Sequence
  Synthetic sequence
PNSERTGHGLRVRFNPLALL

SEQ ID NO: 22 - protein, 20 aa, Artificial Sequence
  Synthetic sequence
GLSKVKEFRHKVRFEGMEPL

SEQ ID NO: 23 - protein, 21 aa, Artificial Sequence
  Synthetic sequence
TFCEDLERKKKAVSFFDDVTV

SEQ ID NO: 24 - protein, 21 aa, Artificial Sequence
  Synthetic sequence
FMATVTKAPKKQIQFADDMQE

SEQ ID NO: 25 - protein, 21 aa, Artificial Sequence
  Synthetic sequence
NAINRYTNTSKTVSFEALPIM

SEQ ID NO: 26 - protein, 21 aa, Artificial Sequence
  Synthetic sequence
APAFLNMRKRKRVTFGEDLSP

SEQ ID NO: 27 - protein, 20 aa, Artificial Sequence
  Synthetic sequence
TCKEDLPQELKIKFLQEAKI

SEQ ID NO: 28 - protein, 21 aa, Artificial Sequence
  Synthetic sequence
AGAGNANSNGKSVKWSEQGGG

SEQ ID NO: 29 - protein, 21 aa, Artificial Sequence
  Synthetic sequence
GRATMSSSNGKSVTWAQNEKS

SEQ ID NO: 30 - protein, 20 aa, Artificial Sequence
  Synthetic sequence
FPVVSFSAGVKVRFLLGGRH

SEQ ID NO: 31 - protein, 19 aa, Artificial Sequence
  Synthetic sequence
VPLGTKFYRVKAVSWNKKV

SEQ ID NO: 32 - protein, 21 aa, Artificial Sequence
  Synthetic sequence
GVEGLCSKEQKRVWFADGILP

SEQ ID NO: 33 - protein, 20 aa, Artificial Sequence
  Synthetic sequence
ELFRDGVNWGRIVAFFEFGG

SEQ ID NO: 34 - protein, 21 aa, Artificial Sequence
  Synthetic sequence
IYLDTPSSGTRRVSFADSFGF

SEQ ID NO: 35 - protein, 21 aa, Artificial Sequence
  Synthetic sequence
ELFRDGVNWGRIVAFFSFGGA

SEQ ID NO: 36 - protein, 21 aa, Artificial Sequence
  Synthetic sequence
ELFQGGPNWGRLVAFFVFGAA

SEQ ID NO: 37 - protein, 21 aa, Artificial Sequence
  Synthetic sequence
FPDTLLGDPGRRVRFFDPLRN

SEQ ID NO: 38 - protein, 21 aa, Artificial Sequence
  Synthetic sequence
IPPMMFDAEQRRVKFINMNGL

SEQ ID NO: 39 - protein, 20 aa, Artificial Sequence
  Synthetic sequence
NSAYICSRRQRVSFGVSEQQ

SEQ ID NO: 40 - protein, 20 aa, Artificial Sequence
  Synthetic sequence
DDTPMRGRQLRVRFATHAAA

SEQ ID NO: 41 - protein, 21 aa, Artificial Sequence
  Synthetic sequence
GAEVAQPREQRRVWFADGILP

SEQ ID NO: 42 - protein, 20 aa, Artificial Sequence
  Synthetic sequence
PEEEEIPANRKIKFSSAPIK

SEQ ID NO: 43 - protein, 20 aa, Artificial Sequence
  Synthetic sequence
SEEEDPAPSRKIHFSTAPIQ

SEQ ID NO: 44 - protein, 21 aa, Artificial Sequence
  Synthetic sequence
SRETANPCSRKKVHFGSIHDA

SEQ ID NO: 45 - protein, 21 aa, Artificial Sequence
  Synthetic sequence
MSQKDGDRPKKKVTFNSSSII

SEQ ID NO: 46 - protein, 21 aa, Artificial Sequence
  Synthetic sequence
YDTRASEPGKKRVRWADLEEK

SEQ ID NO: 47 - protein, 1048 aa, Artificial Sequence
  Synthetic sequence
MSLQFIGLQRRDVVALVNFLRHLTQKPDVDLEAHPKILKKCGEKRLHRRTVLFNELMLWLGYYRELRFHNPDLSSVLEEFEVRCVAVARRGYTYPFGDRGKARDHLAVLDRTEFDTDVRHDAEIVERALVSAVILAKMSVRETLVTAIGQTEPIAFVHLKDTEVQRIEENLEGVRRNMFCVKPLDLNLDRHANTALVNAVNKLVYTGRLIMNVRRSWEELERKCLARIQERCKLLVKELRMCLSFDSNYCRNILKHAVENGDSADTLLELLIEDFDIYVDSFPQSAHTFLGARSPSLEFDDDANLLSLGGGSAFSSVPKKHVPTQPLDGWSWIASPWKGHKPFRFEAHGSLAPAAEAHAARSAAVGYYDEEEKRRERQKRVDDEVVQREKQQLKAWEERQQNLQQRQQQPPPPARKPSASRRLFGSSADEDDDDDDDEKNIFTPIKKPGTSGKGAASGGGVSSIFSGLLSSGSQKPTSGPLNIPQQQQRHAAFSLVSPQVTKASPGRVRRDSAWDVRPLTETRGDLFSGDEDSDSSDGYPPNRQDPRFTDTLVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTASQNTVSTTPRRPSTPRAAVTQTASRDAADEVWALRDQTAESPVEDSEEEDDDSSDTGSVVSLGHTTPSSDYNNDVISPPSQTPEQSTPSRIRKAKLSSPMTTTSTSQKPVLGKRVATPHASARAQTVTSTPVQGRLEKQVSGTPSTVPATLLQPQPASSKTTSSRNVTSGAGTSSASSARQPSASASVLSPTEDDVVSPATSPLSMLSSASPSPAKSAPPSPVKGRGSRVGVPSLKPTLGGKAVVGRPPSVPVSGSAPGRLSGSSRAASTTPTYPAVTTVYPPSSTAKSSVSNAPPVASPSILKPGASAALQSRRSTGTAAVGSPVKSTTGMKTVAFDLSSPQKSGTGPQPGSAGMGGAKTPSDAVQNILQKIEKIKNTEE

(19) United States
(12) Patent Application Publication
(10) Pub. No.: US 2024/0082235 A1
(43) Pub. Date: Mar. 14, 2024

(54) METHOD FOR TREATING OR INHIBITING CYTOMEGALOVIRUS INFECTION USING SMALL MOLECULES TARGETING PROTEIN PHOSPHATASE 1

(71) Applicant: HOWARD UNIVERSITY, Washington, DC (US)

(72) Inventors: Sergei NEKHAI, McLean, VA (US); Christoph STEININGER, Vienna (AT)

(73) Assignee: HOWARD UNIVERSITY, Washington, DC (US)

(21) Appl. No.: 18/267,579

(22) PCT Filed: Dec. 15, 2021
(86) PCT No.: PCT/US2021/063544

§ 371 Date: Jun. 15, 2023

Related U.S. Application Data
(60) Provisional application No. 63/126,202, filed on Dec. 16, 2020.

(51) Int. Cl. A61K 31/473; A61P 31/20

(52) U.S. Cl. CPC A61K 31/473; A61P 31/20

(57) ABSTRACT

A method for treating or inhibiting cytomegalovirus infection, including administrating an effective amount of a class of small molecules targeting protein phosphatase 1 (PP1) to a subject in need thereof. A method for inhibiting replication of cytomegalovirus, including contacting cytomegalovirus or cells containing the cytomegalovirus with a class of small molecules targeting protein phosphatase 1 (PP1).

### TECHNICAL FIELD

Methods pertain to treatment or inhibition of cytomegalovirus infection using a class of small molecules targeting protein phosphatase 1 (PP1). A method for inhibiting replication of cytomegalovirus using a class of small molecules targeting protein phosphatase 1 (PP1).

### BACKGROUND

Protein phosphatase 1 (PP1) is essential for cellular processes such as protein synthesis and cell cycle progression. PP1 functions as a holoenzyme composed of a highly conserved catalytic subunit (PP1c) complexed with one or two variable regulatory proteins such as NIPP1, PNUTS, Sds22 (Peti, W., et al, “Structural basis for protein phosphatase 1 regulation and specificity”, The FEBS journal 280, 2013, 596-611.). PP1 binds to its regulatory subunits mainly via an “RVxF” motif that is accommodated to a hydrophobic groove on the PP1c surface. Multiple viruses use this binding groove to relocate PP1 to the nucleus or regulate PP1 activity for propagation of viral replication.

Recently, a panel of PP1-targeting small molecule compounds that bind RVxF-accommodating groove of PP1 (Ammosova, T. et al, “Small molecules targeted to a non-catalytic “RVxF” binding site of protein phosphatase-1 inhibit HIV-1”. PloS one 7, e39481, 2012a) and C-terminal groove of PP1 (Ammosova, T. et al, “Protein Phosphatase 1-Targeting Small-Molecule C31 Inhibits Ebola Virus Replication”, J Infect Dis 218, S627-S635, 2018) has been developed. The lead compound 1E7-03 inhibits several viruses that utilize PP1, including HIV-1 (Ammosova, T. et al., “1E7-03, a low MW compound targeting host protein phosphatase-1, inhibits HIV-1 transcription”, British journal of pharmacology 171, 5059-5075, 2014b), Ebola and Marburg viruses (Ilinykh, P. A. et al, 2014. “Role of protein phosphatase 1 in dephosphorylation of Ebola virus VP30 protein and its targeting for the inhibition of viral transcription”, The Journal of biological chemistry, 289, 22723-22738, 2014; Tigabu, B. et al, “Phosphorylated VP30 of Marburg Virus Is a Repressor of Transcription”, Journal of virology 92, 2018) and Rift Valley fever virus.

### SUMMARY OF THE INVENTION

The present inventors have conducted an extensive research and have discovered that by targeting PP1, they can achieve inhibition of CMV and also suppression of adaptive immunity.

The discovery that targeting PP1 with the compounds described herein can inhibit transcription and replication of CMV is surprising because the mechanism of replication of EBOV, HIV-1 and CMV are very different. HIV-1, EBOV and CMV use different viral proteins and different host proteins to replicate. The HIV-1 virus is relatively less contagious unless is introduced through a blood transfusion or sexual routes. HIV-1 has a short acute infection period which quickly subsides and is followed by a long latent period during which CD4+ T cells are gradually being destroyed. It primarily infects T-lymphocytes and macrophages of a mammalian system. The HIV-1—mediated destruction of T-lymphocytes adversely impacts the immune system. HIV-1 mutates at a high rate during replication which leads to drug resistance. HIV-1 is a retrovirus, meaning that it uses a reverse transcriptase to convert its RNA within the virion into double stranded DNA, which is then integrated into the host DNA in the nucleus and transcribed by host RNA polymerase II. In contrast, the EBOV primarily infects endothelial cells, mononuclear phagocytes, and hepatocytes, has a relatively short incubation period, is extremely contagious, and its genes are highly conserved during replication. EBOV is a negative-sense RNA virus which transcription and replication occurs in the cytoplasm and is mediated by viral polymerase, protein L. Human cytomegalovirus (CMV) is a member of herpesviruses family which contains a large double stranded DNA genome and encodes its own RNA polymerase and also uses host RNA polymerase for replication, based on fundamentally different principles compared to the RNA viruses mentioned above. CMV hijacks PP1 from the host cell and incorporates it into the tegument of mature viral particles. No other virus known thus far carries PP1 in its own viral particle. Despite these fundamental differences between HIV-1, EBOV and CMV (including their different target cells and usage of host resources), surprisingly PP1 regulates replication of HIV-1, EBOV and also CMV. The compounds described herein can be used to inhibit replication of CMV and block expression of several viral genes. While PP1 is a complex dimer enzyme participating in a wide range of cellular functions, there has been no reason to believe that PP1 is incorporated into CMV virions and treatment with compounds which are effective for HIV-1 and EBOV inhibition, will also be effective to inhibit CMV replication.

In one aspect, described herein are methods for treating or inhibiting cytomegalovirus infection, comprising administrating an effective amount of a compound of formula (I) or a pharmaceutically acceptable salt thereof to a subject in need thereof:

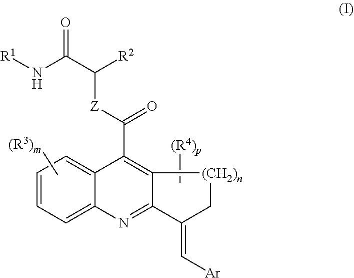

wherein n is 1 or 2;
Ar is phenyl or thienyl, and is optionally substituted;
each R¹ is independently R⁶, C(O)R⁶, C(O)—OR⁶, or C(O)N(R⁶)₂;
R² is H or optionally substituted C1-C6 alkyl, or a group of formula —C(O)NH—R¹;
R³ is independently at each occurrence selected from halo, NO₂, CN, R, OR, NR₂, S(O)_{q}R, COOR, and CONR₂, where each R is independently H, C1-C4 alkyl, or C1-C4 haloalkyl;
m is 0-4;
R⁴ is R⁶, halo, ═O, COOR⁶, CON(R⁶)₂, S(O)_{q}R⁶, N(R⁶)₂, or OR⁶;
p is 0-2;
each q is independently 0-2;
Z is O or NR⁵;
R⁵ is R⁶ or C(O)R⁶; and
R⁶ is independently at each occurrence selected from H, C1-C6 alkyl, C5-C6 aryl, and (C5-C6-aryl)-C1-C6 alkyl, where each alkyl and aryl is optionally substituted;
provided that n is 2 when Z is O and Ar represents para-halophenyl.

Also described herein are methods of inhibiting replication of CMV with the compounds of general formula (I) or a pharmaceutically acceptable salt thereof.

In one aspect, the compound of general formula (I) is 1E7-03 represented by the following formula:

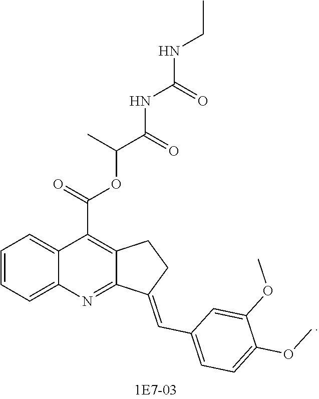

The present inventors have discovered that 1E7-03 and its derivatives efficiently inhibit CMV replication in vitro.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows sequence alignment of validated human PP1 binders through “RVXF” motif. Amino acid colors represent hydrophobicity in the left panel. In the right panel, Sequence LOGOs of the 3 most common PP1 binding motifs (RVXF, MyPhoNE and SILK), generated from human PP1 binding proteins using MEME, are shown.

FIG. 2 shows sequence of CMV UL32/pp150 protein with indicated SILK motif (blue) and RVXF motif (yellow).

FIG. 3 shows structure of 1E7-03.

FIG. 4A shows binding of PP1 fused to a large bit to cdNIPP1 fused to a small bit in split NanoBit system. FIG. 4B shows effect of 1E7-03 on PP1-cdNIPP1 interaction in split NanoBit system.

FIGS. 5A-5C show inhibition of CMV replication by 1E7-03, a PP1-targeting small molecule. FIG. 5A shows plaque assays of human foreskin fibroblasts infected with CMV shows concentration-dependent inhibition of CMV replication; percentage compared to untreated-control. FIG. 5B shows that the expression of CMV immediate early proteins is reduced by 1E7-03. FIG. 5C shows viral mRNA expression that was measured in infected human foreskin fibroblasts by RT-qPCR, showing fold-change compared to infected cells treated with vehicle only. Titration of 1E7-03 measuring IE1 expression and quantification of IE1, IE2 and UL32 cDNA shows no effect on viral transcription.

### DETAILED DESCRIPTION OF THE INVENTION

PP1 is important for human cytomegalovirus (CMV) but the mode of action and interaction between PP1 and CMV proteins has not been completely understood. CMV activation often occurs in patients with solid organ transplants when immunosuppressive drugs are administered. On the other hand, suppression of T cell responses in hematopoietic bone marrow or solid organ transplant recipients or in patients with AIDS may cause overt CMV replication and disease. Hence, CMV has an enormous impact on the overall immune profile of the infected patients, especially with respect to CD8+ T cells.

The present inventors have discovered that 1E7-03 and its derivatives efficiently inhibit CMV replication in vitro.

In one aspect, described herein are methods for treating or inhibiting cytomegalovirus, comprising administrating an effective amount of a compound of formula (I) or a pharmaceutically acceptable salt thereof to a subject in need thereof:

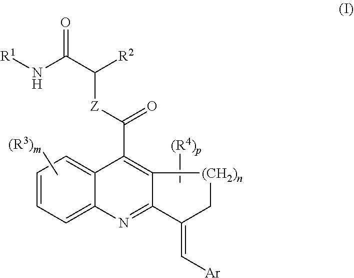

wherein n is 1 or 2;
Ar is phenyl or thienyl, and is optionally substituted;
each R¹ is independently R⁶, C(O)R⁶, C(O)—OR⁶, or C(O)N(R⁶)₂;
R² is H or optionally substituted C1-C6 alkyl, or a group of formula —C(O)NH—R¹;
R³ is independently at each occurrence selected from halo, NO₂, CN, R, OR, NR₂, S(O)_{q}R, COOR, and CONR₂, where each R is independently H, C1-C4 alkyl, or C1-C4 haloalkyl;
m is 0-4;
R⁴ is R⁶, halo, ═O, COOR⁶, CON(R⁶)₂, S(O)_{q}R⁶, N(R⁶)₂, or OR⁶;
p is 0-2;
each q is independently 0-2;
Z is O or NR⁵;
R⁵ is R⁶ or C(O)R⁶; and
R⁶ is independently at each occurrence selected from H, C1-C6 alkyl, C5-C6 aryl, and (C5-C6-aryl)-C1-C6 alkyl, where each alkyl and aryl is optionally substituted;
provided that n is 2 when Z is O and Ar represents para-halophenyl.

In some embodiments, Z is NR⁵. In such embodiments, R⁵ is selected from H and —C(O)R′, where R′ is a C1-C4 alkyl or C1-C4 haloalkyl. In other embodiments, Z is O or NH; preferably Z is O.

In some embodiments, Ar is phenyl, which is optionally substituted. Preferably, wherein n is 1, Ar is not 4-halophenyl.

In other embodiments, Ar is thienyl, which can be substituted. Theienyl can be attached at either position 2 or position 3 of the thiophene ring. In some embodiments, Ar is 2-thienyl, and is optionally substituted. In other embodiments, Ar is optionally substituted 3-thienyl.

In some embodiments, n is 1. In some embodiments, n is 2.

In some embodiments, R² is H or C1-C4 alkyl or C1-C4 haloalkyl. Preferably, R² is H, methyl or ethyl.

In some embodiments, when m is not 0, at least one R³ is halo, C1-C4 alkyl, or C1-C4 haloalkyl.

In some embodiments, p is 0. In other embodiments, p is 1-2.

Where p is not 0, in some embodiments at least one R⁴ is ═O, C1-C4 alkyl, or C1-C4 haloalkyl.

In some embodiments, R¹ is an optionally substituted C1-C6 alkyl. In other embodiments, R¹ is C(O)R⁶. In other embodiments, R¹ is C(O)NHR⁶.

The compounds of formula (I) readily form acid addition salts. In some embodiments, the compound of formula (I) is an acid addition salt. In many embodiments, the acid addition salt is a pharmaceutically acceptable salt.

Also described herein are methods of inhibiting replication of CMV with the compounds of general formula (I) or a pharmaceutically acceptable salt thereof.

As used herein, the terms “alkyl,” “alkenyl,” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclo-pentylethyl, 2-propenyl, 3-butynyl, and the like. The total number of carbon atoms in each such group is sometimes described herein. For example, a group containing up to ten carbon atoms is expressed as “1-10 C,” “C1-C10” or “C1-10.” When heteroatoms (N, O and S typically) are substituted for carbon atoms, such as in heteroalkyl groups, the numbers describing the group (e.g., C1-C6) represent the sum of the number of carbon atoms in the group plus the number of such heteroatoms that are substituted for carbon atoms in the ring or chain being described.

Typically, the alkyl, alkenyl and alkynyl substituents of the compounds described herein are 1-10 C(alkyl) or 2-10 C (alkenyl or alkynyl), preferably 1-8 C (alkyl) or 2-8 C (alkenyl or alkynyl), and in some aspects 1 -4 C (alkyl) or 2-4 C (alkenyl or alkynyl). A single group can include more than one type of multiple bond or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR, SO₂R′, NR′CONR′, NR′COOR′, NR′COR′, CN, COOR, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a nonaromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acryl or heteroacyl group.

“Aromatic” moiety or “aryl” moiety refers to a monocyclic or fused bicyclic moiety having the well-known characteristics of aromaticity; examples include phenyl and naphthyl. :Aryl” can include aromatic ring systems containing only carbon as well as aromatic ring systems containing one or more heteroatoms (O, N or S) as ring members. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any mono cyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contains 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenyl ethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, 0 and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)2- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R7 is alkyl, this alkyl may optionally be substituted by the remaining substituents listed as embodiments for R7 where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its substituted with fluorine atoms at any or all of its available valences, for example. In some embodiments, where no number of substituents is specified, the number is preferably 0-2.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Halo,” as used herein includes fluoro, chloro, bromo and iodo. Fluoro and chloro are often preferred.

“Haloalkyl” as used herein includes alkyl groups having one or more halogen substituents. Examples include trifluoromethyl, 2,2,2-trifluoroethyl, 2-chloroethyl, 2-fluoroethyl, and the like.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

Where isomers are possible, the invention includes each individual isomer as well as mixtures of isomers. Where a chiral center is present, the invention includes each individual enantiomer at the chiral center as well as mixtures of enantiomers, including racemic mixtures.

The compounds described herein can be prepared using well-known reactions, starting from available starting materials such as 1,2,3,4-tetrahydroacridine-9-carboxylic acid as summarized in Scheme 1. This acid can readily be converted to an ester or an amide to provide compounds wherein Z is O or N, respectively, using standard conditions that are well known in the art. The wide array of available alcohols and amines enables one to synthesize many compounds with various R¹ and R² groups incorporated therein. Once an ester or amide is formed from the carboxylate, the intermediate ester or amide can be condensed with various available aldehydes to introduce the “Ar—CH═” group on the saturated ring, using a base such as potassium tert-butoxide in a polar, aprotic solvent such as DMSO, DMF, DME, or THF, or in a non-nucleophilic protic solvent such as t-butanol. It is also possible to form a hindered ester of the starting carboxylic acid, such as a t-butyl ester, and condense the acridine ester with an aldehyde as described, then hydrolyze the ester to make an intermediate carboxylic acid compound having the Ar—CH═ group in place. This intermediate can then be coupled to various available or readily accessible alcohols or amines to produce the products of formula (I). Methods for such coupling reactions are well known in the art.

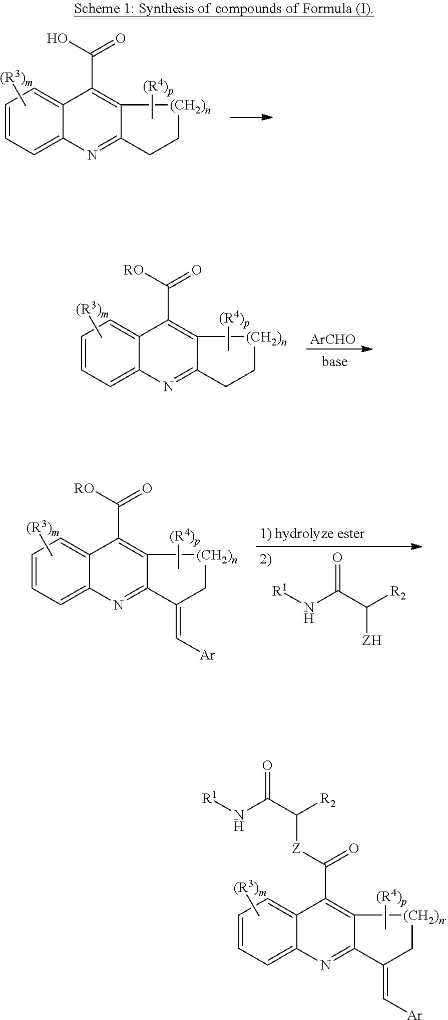

The compounds of formula I and other compounds described herein may be administered by oral, parenteral (e.g., intramuscular, intraperitoneal, intravenous, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. Methods and formulations for each of these routes of administration are within the knowledge and expertise of a person of ordinary skill in the art

The compounds of formula I and other compounds described herein may form hydrates or solvates, which are included in the scope of the claims. When the compounds of formula I and other compounds described herein exist as regioisomers, configurational isomers, conformers, or diasteroisomeric forms, all such forms and various mixtures thereof are included in the generic formulas. It is possible to isolate individual isomers using known separation and purification methods, if desired. For example, when a compound of formula I is a racemate, the racemate can be separated into the (S)-compound and (R)-compound by optical resolution. Individual optical isomers and mixtures thereof are included in the scope of the generic formula.

The compounds of the invention can be used in their neutral form or as a salt. The compounds of Formula I and other compounds described herein readily form acid addition salts, and in some embodiments, the acid addition salts are preferable for use in the methods and pharmaceutical compositions of the invention. Formation of such salts is within the ordinary level of skill in the art and can be achieved by contacting a compound of Formula I or other compounds described herein with a suitable acid. The salt used can be any stable salt; in some embodiments, the acid is selected to provide a pharmaceutically acceptable salt. Examples of pharmaceutically acceptable salts are organic acid addition salts formed with acids that form a physiological acceptable anion, for example, tosylate, methanesulfonate, besylate, acetate, formate, citrate, malonate, tartrate, succinate, benzoate, ascorbate, a-ketoglutarate, lactate, and a-glycerophosphate. Suitable inorganic salts may also be formed, including hydrochloride, sulfate, bisulfate, phosphate, nitrate, hydrobromide, and the like.

Compositions are provided that include a pharmaceutically acceptable carrier or diluent and an effective amount of a compound of Formula I or other compounds described herein. The pharmaceutical compositions preferably comprise at least one acceptable diluent or excipient other than water, methanol, ethanol, or DMSO. In some embodiments, the pharmaceutical composition comprises at least one excipient selected from a buffer, saline, and a mono- or di-saccharide.

A compound of formula I and other compounds described herein may be administered alone or as an admixture with a pharmaceutically acceptable carrier (e.g., solid formulations such as tablets, capsules, granules, powders, etc.; liquid formulations such as syrups, injections, etc.) and may be orally or non-orally administered. Examples of non-oral formulations include injections, drops, suppositories, and pessaries.

In the treatment or prevention of conditions in a human subject, an appropriate dosage level will generally be about 0.01 to 50 mg per kg patient body weight per day which can be administered in single or multiple doses. Preferably, it is believed the dosage level will be from about 0.1 to about 10 mg/kg per day. It will be understood that the specific dose level and frequency of dosage for any particular patient may be varied and will depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

In one embodiment, a compound is administered systemically (e.g., orally) in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may be varied and may conveniently be between about 2 to about 60% of the weight of a given unit dosage form. The amount of active compound in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active compound may be incorporated into sustained-release preparations and devices.

The active compound also may be administered intravenously or intraperitoneally by infusion or injection. Solutions of the active compound or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The compound is sometimes prepared as a polymatrix-containing formulation for such administration (e.g., a liposome or microsome). Liposomes are described for example in U.S. Pat. No. 5,703,055 (Feigner, et al.) and Gregoriadis, Liposome Technology vols. I to II (2nd ed. 1993).

The pharmaceutical dosage forms suitable for injection or infusion can include sterile aqueous solutions or dispersions or sterile powders comprising the active ingredient that—are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in liquid form. Compounds often are administered as compositions or formulations, in combination with a dermatologically acceptable carrier, which may be a solid or liquid. Examples of useful dermatological compositions used to deliver compounds to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortman (U.S. Pat. No. 4,820,508).

Compounds may be formulated with a solid carrier, which can include finely divided solids such as talc, clay, microcrystalline cellulose, silica, or alumina and the like. Useful liquid carriers include water, alcohols or glycols or water/alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the compound in a liquid composition often is from about 0.1 wt % to about 25 wt %, sometimes from about 0.5 wt % to about 10 wt %. It is believed the concentration in a semi-solid or solid composition such as a gel or a powder often is about 0.1 wt % to about 5 wt %, sometimes about 0.5 wt % to about 2.5 wt. A compound composition may be prepared as a unit dosage form, which is prepared according to conventional techniques known in the pharmaceutical industry. In general terms, such techniques include bringing a compound into association with pharmaceutical carriers) and/or excipient(s) in liquid form or finely divided solid form, or both, and then shaping the product if required. The compound composition may be formulated into any dosage from, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The compound of formula I and other compounds described herein may be administered in combination with other medicine (for example, well-known agents for treating cytomegalovirus infection) for the purposes of: (1) supplementing and/or enhancing therapeutic effect, (2) improving the kinetics, improving absorption, and reducing the dose; and/or (3) eliminating the adverse reaction of the compound.

### EXAMPLES

The present invention is explained below in further detail with reference to Examples. However, the scope of the invention is not limited to these Examples. Unless otherwise specified all parts and percentages are by weight, and reported measurements and other data were obtained under ambient conditions.

CMV Proteins Contain PP1-Binding Motifs.

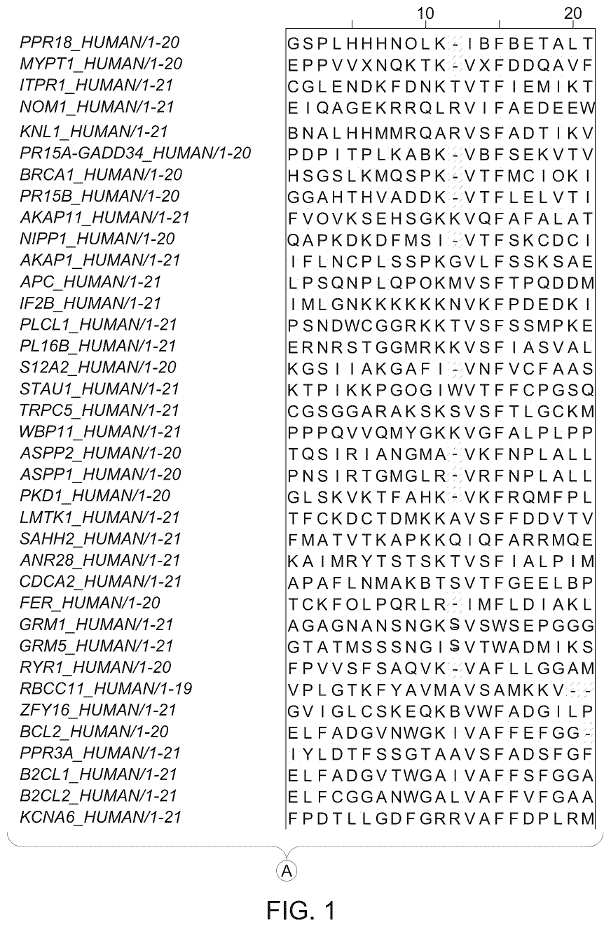

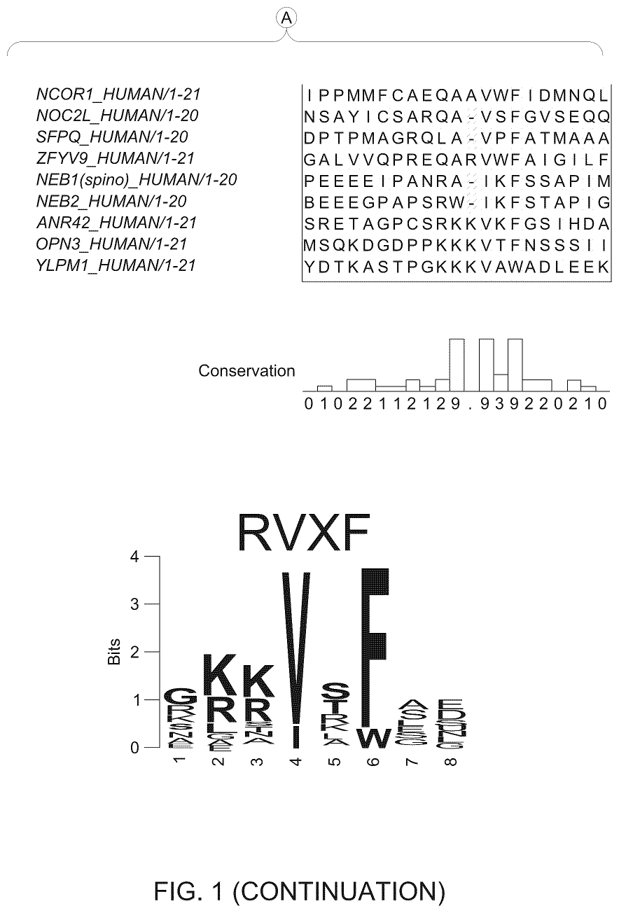

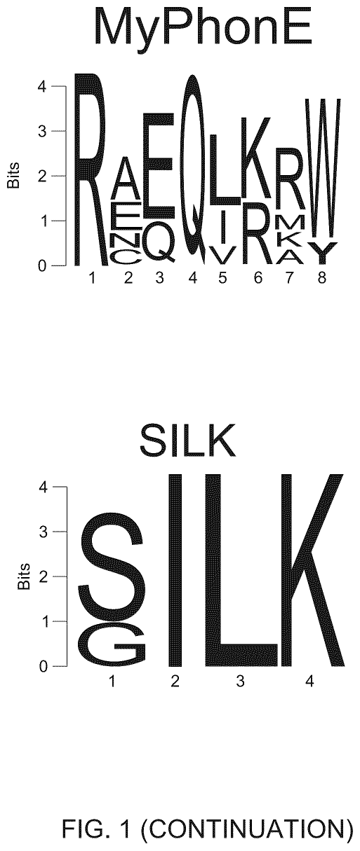

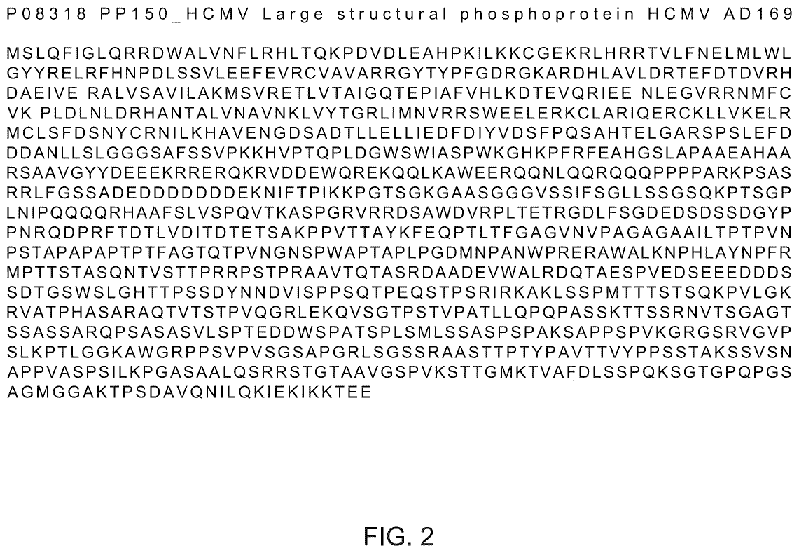

22 CMV tegument proteins (as defined in Kalejta, R. F. “Tegument proteins of human cytomegalovirus. Microbiology and molecular biology reviews”, MMBR 72, 249-265, 2008, table of contents) contain one or more putative RVxF or non-canonical viral “QVxF” binding motif (FIG. 1) (Ammosova, T. et al, “Nuclear targeting of protein phosphatase-1 by HIV-1 Tat protein”, The Journal of biological chemistry 280, 36364-36371, 2005). 1 CMV tegument contains an additional PP1 binding motif (FIG. 2, see an example of UL32/pp150 protein with SILK and RVxF motif).

Inhibition of CMV by PP1-Targeting Small Molecules.

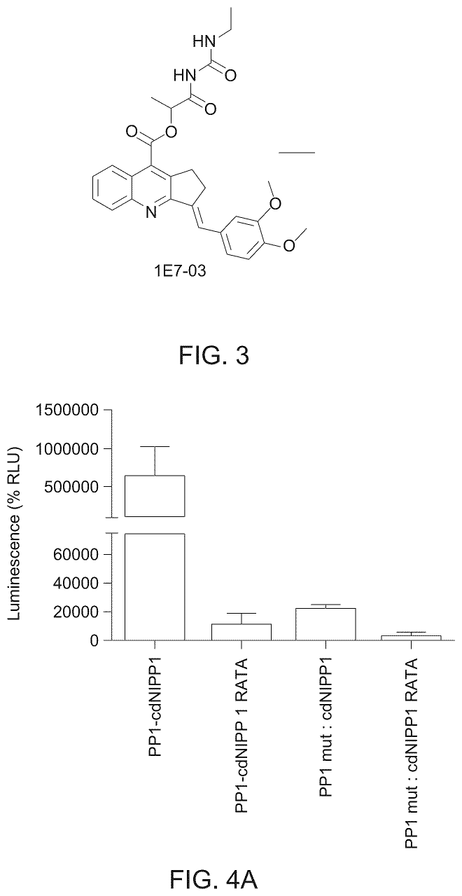

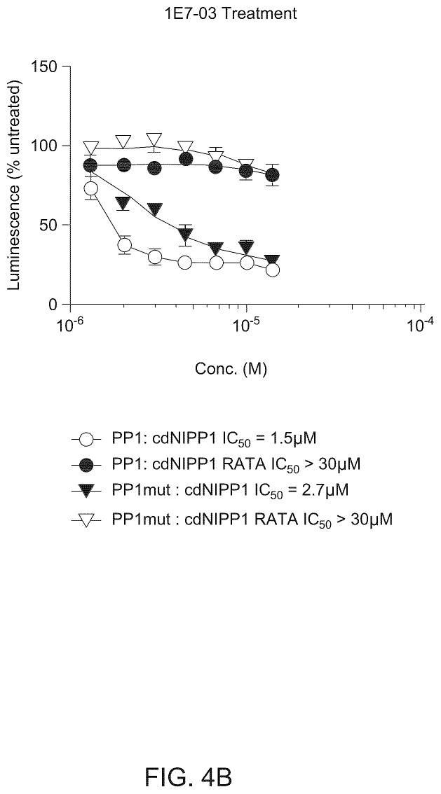

Previous small molecule screens, conducted by Inventor's group, led to the discovery of compound 1E7-03 (FIG. 3) which disrupted the interaction of PP1 with peptides containing an RVxF sequence in vitro (Ammosova, T. et al, “1E7-03, a low MW compound targeting host protein phosphatase-1, inhibits HIV-1 transcription”, Br. J. Pharmacol. 171, 5059-5075, 2014a; Ammosova, T. et al, “Small Molecules Targeted to a Non-Catalytic “RVxF” Binding Site of Protein Phosphatase-1 Inhibit HIV-1”, PloS one 7, e39481, 2012b). Surface plasmon resonance (SPR) confirmed that 1E7-03 binds to PP1 (K_{D}=6.2 μM) (Lin, X. et al, “Inhibition of HIV-1 infection in humanized mice and metabolic stability of protein phosphatase-1-targeting small molecule 1E7-03”, Oncotarget 8, 76749-76769, 2017). To this end, split Nanobit protein:protein interaction assays were developed for screening of PP1-targeted small molecules. The Nanobit system was recently introduced by Promega. This system utilizes NanoLuc, an ATP—independent luciferase that uses a novel substrate (furimazine) to produce high intensity luminescence. PP1 was fused to the Small BiT (SmBiT; 11 amino acids) and PP1-interacting proteins were fused to the large BiT (LgBiT; 17.6 kDa). Split NanoBit assay system was tested with wild type (WT) PP1 and C-terminally mutated PP1 (PP1 mut) binding to WT cdNIPP1 or mutant cdNIPP1 with an RVxF mutation (cdNIPP1 RATA) (FIG. 4) (Lin, X. et al, “Targeting the Non-catalytic RVxF Site of Protein Phosphatase-1 With Small Molecules for Ebola Virus Inhibition”, Frontiers in microbiology 10, 2019). NIPP1 is one of the major nuclear interactors of protein phosphatase PP1 and occupies the RVxF site on PP1 (O'Connell, N. et al, “The Molecular Basis for Substrate Specificity of the Nuclear NIPP1:PP1 Holoenzyme”, Structure 20, 1746-1756, 2012). Both the RATA mutation and mutation in the PP1 C-terminal domain reduced the interaction of PP1 with cdNIPP1 (FIG. 4A). Treatment with compound 1E7-03 inhibited the PP1-cdNIPP1 interaction and PP1mut-cdNIPP1 interaction to a similar extent, but did not have an effect on PP1-cdNIPP1 RATA interaction showing that 1E7-03 primarily acts on the RVxF binding site of PP1 (FIG. 4B). Thus, using split NanoBit system allowed the inventors to confirm that 1E7-03 interacts with the RVxF motif of PP1.

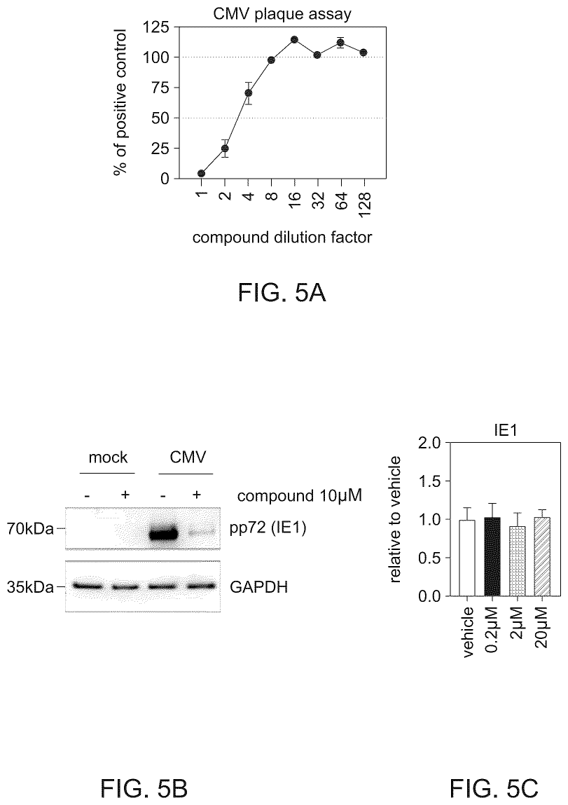

Then, the effect of 1E7-03 and its derivatives on CMV replication was tested. The inventors observed potent CMV inhibition by 1E7-03 that included the inhibition of CMV replication and severely inhibited viral immediate early protein translation (FIG. 5) Inhibition of CMV propagation very likely occurs via novel mechanisms compared to the effect of PP1 manipulation in ssRNA viruses, especially since it is surprising that CMV mRNA transcription is not affected by 1E7-03 (FIG. 5), underlining that the mechanism of action of 1E7-03 is likely substantially different from HIV and EBOV inhibition. First, PP1 modulation might counteract a viral strategy to re-enable protein translation after eIF-2α mediated ribosomal shutdown, as observed in other herpes viruses (Li, Y. et al, 2011. “ICP34.5 protein of herpes simplex virus facilitates the initiation of protein translation by bridging eukaryotic initiation factor 2alpha (eIF2alpha) and protein phosphatase 1”, The Journal of biological chemistry 286, 24785-24792, 2011). Second, deregulation of PP1 might serve as a viral immunoevasion mechanism since PP1 has been shown to be involved in innate immune signaling on multiple layers, e.g. by interfering with TLR signaling or activation of intracellular RNA sensors (Wies, E. et al, “Dephosphorylation of the RNA sensors RIG-I and MDA5 by the phosphatase PP1 is essential for innate immune signaling”, Immunity 38, 437-449, 2013).

In summary, the above observations demonstrate that the PP1-targeting compounds are able to inhibit CMV replication and viral protein production.

While the subject matter disclosed herein has been described in connection with what is presently considered to be practical example embodiments, it is to be understood that the present disclosure is not limited to the disclosed embodiments, and covers various modifications and equivalent arrangements included within the spirit and scope of the appended claims.

What is claimed is:

1. A method for treating or inhibiting cytomegalovirus infection, comprising administrating an effective amount of a compound of formula (I) or a pharmaceutically acceptable salt thereof to a subject in need thereof: 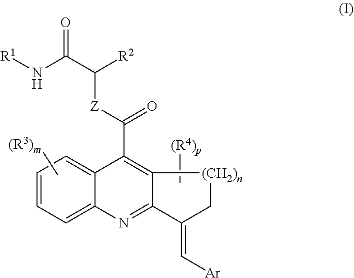
wherein n is 1 or 2;
Ar is phenyl or thienyl, and is optionally substituted;
each R¹ is independently R⁶, C(O)R⁶, C(O)—OR⁶, or C(O)N(R⁶)₂;
R² is H or optionally substituted C1-C6 alkyl, or a group of formula —C(O)NH—R¹;
R³ is independently at each occurrence selected from halo, NO₂, CN, R, OR, NR₂, S(O)_{q}R, COOR, and CONR₂, where each R is independently H, C1-C4 alkyl, or C1-C4 haloalkyl;
m is 0-4;
R⁴ is R⁶, halo, ═O, COOR⁶, CON(R⁶)₂, S(O)_{q}R⁶, N(R⁶)₂, or OR⁶;
p is 0-2;
each q is independently 0-2;
Z is O or NR⁵;
R⁵ is R⁶ or C(O)R⁶; and
R⁶ is independently at each occurrence selected from H, C1-C6 alkyl, C5-C6 aryl, and (C5-C6-aryl)-C1-C6 alkyl, where each alkyl and aryl is optionally substituted;
provided that n is 2 when Z is O and Ar represents para-halophenyl.

2. The method according to claim 1, wherein Z is O.

3. The method according to claim 1, wherein n is 1.

4. The method according to claim 1, wherein R² is H or C₁-C₄ alkyl.

5. The method according to claim 1, wherein Ar is optionally substituted phenyl.

6. The method according to claim 1, wherein the compound is 1E7-03 represented by the following formula:

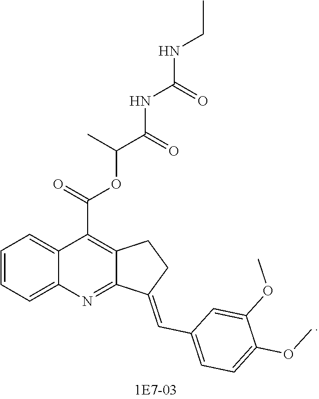 [SMILES: CCNC(=O)NC(=O)C(C)OC(=O)c1c2c(nc3ccccc13)/C(=C/c1ccc(OC)c(OC)c1)CC2]

7. The method according to claim 1, wherein the compound of formula (I) or pharmaceutically acceptable salt thereof is administered orally or parenterally.

8. The method according to claim 1, wherein the compound of formula (I) or pharmaceutically acceptable salt thereof is administered 0.1 to 10 mg per kg patient body weight per day.

9. A method for inhibiting replication of cytomegalovirus, comprising contacting the cytomegalovirus or a cell containing the cytomegalovirus with a compound of formula (I) or a pharmaceutically acceptable salt thereof: 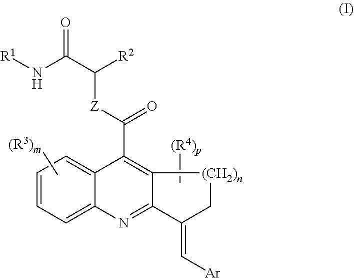
wherein n is 1 or 2;
Ar is phenyl or thienyl, and is optionally substituted;
each R¹ is independently R⁶, C(O)R⁶, C(O)—OR⁶, or C(O)N(R⁶)₂;
R² is H or optionally substituted C1-C6 alkyl, or a group of formula —C(O)NH—R¹;
R³ is independently at each occurrence selected from halo, NO₂, CN, R, OR, NR₂, S(O)_{q}R, COOR, and CONR₂, where each R is independently H, C1-C4 alkyl, or C1-C4 haloalkyl;
m is 0-4;
R⁴ is R⁶, halo, ═O, COOR⁶, CON(R⁶)₂, S(O)_{q}R⁶, N(R⁶)₂, or OR⁶;
p is 0-2;
each q is independently 0-2;
Z is O or NR⁵;
R⁵ is R⁶ or C(O)R⁶; and
R⁶ is independently at each occurrence selected from H, C1-C6 alkyl, C5-C6 aryl, and (C5-C6-aryl)-C1-C6 alkyl, where each alkyl and aryl is optionally substituted;
provided that n is 2 when Z is O and Ar represents para-halophenyl.

10. The method according to claim 9, wherein Z is O.

11. The method according to claim 9, wherein n is 1.

12. The method according to claim 9, wherein R² is H or C₁-C₄ alkyl.

13. The method according to claim 9, wherein Ar is optionally substituted phenyl.

14. The method according to claim 9, wherein the compound is 1E7-03 represented by the following formula:

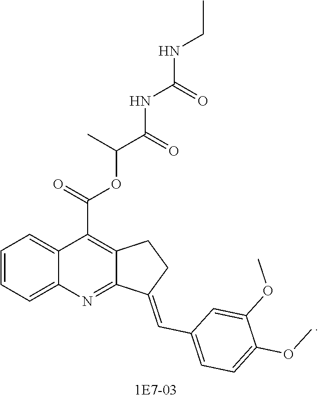 [SMILES: CCNC(=O)NC(=O)C(C)OC(=O)c1c2c(nc3ccccc13)/C(=C/c1ccc(OC)c(OC)c1)CC2]

15. The method according to claim 9, wherein the compound of formula (I) or pharmaceutically acceptable salt thereof is administered orally or parenterally.

16. The method according to claim 9, wherein the compound of formula (I) or pharmaceutically acceptable salt thereof is administered 0.1 to 10 mg per kg patient body weight per day.
